(19) United States
(12) Patent Application Publication
(10) Pub. No.: US 2025/0222228 A1
(43) Pub. Date: Jul. 10, 2025

(54) UNCONSTRAINED SLEEP IMPROVEMENT DEVICE AND SLEEP IMPROVEMENT METHOD USING THE SAME

(71) Applicant: BRLAB INC., Seoul (KR)

(72) Inventors: Jong Min LEE, Seoul (KR); Hyun Bin KWON, Seoul (KR); Il Song LEE, Seoul (KR)

(21) Appl. No.: 19/093,689

(22) Filed: Mar. 28, 2025

Related U.S. Application Data
(63) Continuation of application No. PCT/KR2022/019502, filed on Dec. 2, 2022.

(30) Foreign Application Priority Data
Sep. 30, 2022 (KR) 10-2022-0124875
Sep. 30, 2022 (KR) 10-2022-0124882

(51) Int. Cl. A61M 21/02; A61B 5/00

(52) U.S. Cl. CPC A61M 21/02; A61B 5/4806; A61B 2562/0247

(57) ABSTRACT

The present disclosure relates to an unrestrained sleep improvement device and a sleep improvement method using the same, and more particularly, to a sleep improvement device that senses biometric information items from a user on a bed or mattress, identifies the user's sleep state from the sensed biometric information items, and generates and provides stimulation for inducing a change in the user's biometric signal according to the identified sleep state, and a sleep improvement method using the same.

### CROSS-REFERENCE TO RELATED APPLICATIONS

This application is a Continuation of Application No. PCT/KR2022/019502, filed on Dec. 22, 2021, which in turn claims the benefit of Korean Patent Applications No. 10-2022-0124872, filed on Sep. 30, 2022, and No. 10-2022-0124882, filed on Sep. 30, 2022. The entire disclosures of all these applications are hereby incorporated by reference.

### TECHNICAL FIELD

The present disclosure relates to an unrestrained sleep improvement device and a sleep improvement method using the same, and more particularly, to a sleep improvement device that senses biometric information items from a user on a bed or mattress, identifies the user's sleep state from the sensed biometric information items, and generates and provides stimulation for inducing a change in the user's biometric signal according to the identified sleep state, and a sleep improvement method using the same.

### BACKGROUND ART

As interest in ‘good sleep’ increases, the sleep industry, especially the industry that provides various devices or services using sleep science, is growing significantly. Many devices and services are being commercialized to improve sleep quality, such as sleep care services that provide guidance on sleep environment, habits, and posture through consulting with experts, and services that monitor a sleep state by sensing the user's breathing sounds, or the like when wearing a wearable device.

Meanwhile, it is common sense that a good sleep comes when the user lies comfortably on a mattress or bed, but most people in the sleep industry or sleep science field still use constrained devices to identify the user's sleep state. For example, monitoring devices in the form of a headgear that can be worn on the head, a monitoring device that is inserted into the nose, a monitoring device that is mounted on the fingertip, and a monitoring device that is attached to the heart may be understood as one type of the constrained device.

In order to identify the sleep state, it is necessary to measure relatively accurate user biometric information, but, in order to measure accurate information, it is inevitable to use sensors that come into direct contact with the user's body, and thus so-called constrained devices have been used in the past. However, the constrained devices inevitably come into contact with the user's body, and as a result, there is a very high possibility that they will cause discomfort during sleep.

The present disclosure has been proposed in consideration of such problems, and aims to provide a sleep improvement device that can identify a sleep state without constraining the user's body by installing sensing means in a mattress-shaped product, and a sleep improvement method using the same.

### DISCLOSURE OF INVENTION

### Technical Problem

An aspect of the present disclosure is to acquire a user's biometric information without any constraints, thereby making it possible to basically identify a sleep state without disturbing the user's sleep.

Furthermore, an aspect of the present disclosure is to extract meaningful information items from acquired biometric information and to accurately identify the user's sleep state from the extracted information.

Furthermore, an aspect of the present disclosure is to generate and provide stimulation that can induce a change in the user's biometric signal to improve the identified sleep state.

Meanwhile, technical problems of the present disclosure are not limited to the above-mentioned problems, and other technical problems which are not mentioned herein will be clearly understood by those skilled in the art from the description below.

### Technical Solution

In order to solve the foregoing problems, an unrestrained sleep improvement device according to the present disclosure may include at least one layer including a plurality of components; a first sensing unit provided on the layer to acquire a vibration signal from a user; a second sensing unit provided on the layer to acquire a pressure signal according to the user's movement; a calculation unit that determines the user's sleep state or autonomic nervous system state from the signals acquired by the first and second sensing units; and a stimulation unit that generates stimulation to induce a change in the user's biometric signal according to the sleep state or autonomic nervous system state.

Furthermore, in the unrestrained sleep improvement device, at least one of the first sensing unit or the second sensing unit may be provided within an area of 1/10 to ⅓ from the top based on a vertical length of the layer.

Furthermore, in the unrestrained sleep improvement device, at least one of the first sensing unit or the second sensing unit may include a curved surface that is curved in an upward or downward direction of the layer.

Furthermore, in the unrestrained sleep improvement device, the first or second sensing unit may include a wave-shaped belting member and a plurality of sensors arranged on the member.

Furthermore, in the unrestrained sleep improvement device, the calculation unit may process a vibration signal acquired by the first sensing unit to extract a plurality of valid signals, and generate biometric information on the user from the extracted valid signals.

Furthermore, in the unrestrained sleep improvement device, the calculation unit may record a correlation between the vibration signal acquired by the first sensing unit and the pressure signal acquired by the second sensing unit per unit time to create a database.

Furthermore, in the unrestrained sleep improvement device, the unrestrained sleep improvement device may be a mattress, the device further including a touch-type input pad, which is provided on a side surface of the mattress to receive an touch input from the user.

Meanwhile, a method of improving a user's sleep using an unrestrained sleep improvement device according to another embodiment of the present disclosure may include receiving a vibration signal from a first sensing unit; receiving a pressure signal from a second sensing unit; determining the user's sleep state or autonomic nervous system state from the signals acquired by the first and second sensing units; and generating stimulation to induce a change in the user's biometric signal according to the sleep state or autonomic nervous system state.

### Advantageous Effects

According to the present disclosure, there is an effect in which biometric information items for identifying a sleep state can be acquired without disturbing a user from getting a deep sleep. That is, the user's biometric information items may be obtained in an unrestrained environment, thereby allowing to generate stimulation to improve the user's sleep quality, generate stimulation to induce autonomic nervous system activity, and identify a sleep state.

In addition, according to the present disclosure, various types of meaningful information items may be extracted from acquired biometric information items, thereby having an effect that can identify a sleep state in a more multi-faceted manner.

In addition, according to the present disclosure, there is an effect that do not rely on drugs by improving a sleep state, and can safely and fundamentally care for the user's biological rhythm in a long term.

In particular, according to the present disclosure, there is an effect that can improve diseases or disorders such as sleep apnea, insomnia, PTSD, and mild cognitive impairment.

In addition, according to the present disclosure, there is an effect that can simultaneously identify sleep states of two or more users, or can provide stimulation for improving the sleep states.

Meanwhile, the effects of the present disclosure may not be limited to the above-mentioned effects, and other technical effects which are not mentioned herein will be clearly understood by those skilled in the art from the description below.

### BRIEF DESCRIPTION OF THE DRAWINGS

FIG. 1a and FIG. 1b show an unrestrained sleep improvement device according to the present disclosure.

FIG. 2 shows another sleep improvement device according to the present disclosure, in which internal components of a sleep improvement device capable of acquiring biometric signals for two users are conceptually shown.

FIG. 3 is a drawing for explaining a shape and location of a sensing unit.

FIG. 4 is a drawing for explaining another type of a sensing unit shape (sinusoidal shape).

FIG. 5 shows a third sensing unit that can be adhered to a user's skin surface.

FIG. 6 shows a fourth sensor unit that can be worn on a user's ear.

FIG. 7 shows an original signal acquired by a first sensing unit and information items that can be extracted therefrom.

FIG. 8 shows an original signal acquired by a second sensing unit and a user's sleeping posture estimated therefrom.

FIG. 9 shows a configuration in which three or more plurality of sensing units are provided on a layer.

FIG. 10 shows an example of a touch-type input pad that can receive a touch input from a user.

FIG. 11 shows a sleep improvement method in sequence.

### BEST MODE FOR CARRYING OUT THE INVENTION

The details of the objects and technical configurations of the present disclosure and operational effects thereof will be more clearly understood from the following detailed description based on the accompanying drawings appended hereto. Hereinafter, embodiments according to the present disclosure will be described in detail with reference to the accompanying drawings.

Embodiments disclosed herein should not be interpreted as limiting or used to limit the scope of the present disclosure. It is apparent for those skilled in the art that a description including embodiments herein has various applications. Therefore, any embodiments described in the detailed description of the present disclosure are illustrative for better understanding of the present disclosure and are not intended to limit the scope of the present disclosure to the embodiments.

Functional blocks illustrated in the drawings and described hereunder are only examples of possible implementations. In other implementations, other functional blocks may be used without departing from the concept and scope of the detailed description. Furthermore, one or more functional blocks of the present disclosure are illustrated as separate blocks, but one or more of the functional blocks of the present disclosure may be a combination of various hardware and software elements that execute the same function.

In addition, an expression that some elements are “included” is an expression of an “open type”, and the expression simply denotes that the corresponding elements are present, but should not be construed as excluding additional elements.

Moreover, in case where it is mentioned that one element is “connected” or “coupled” to the other element, it should be understood that one element may be directly connected to the other element, but another element may be present therebetween.

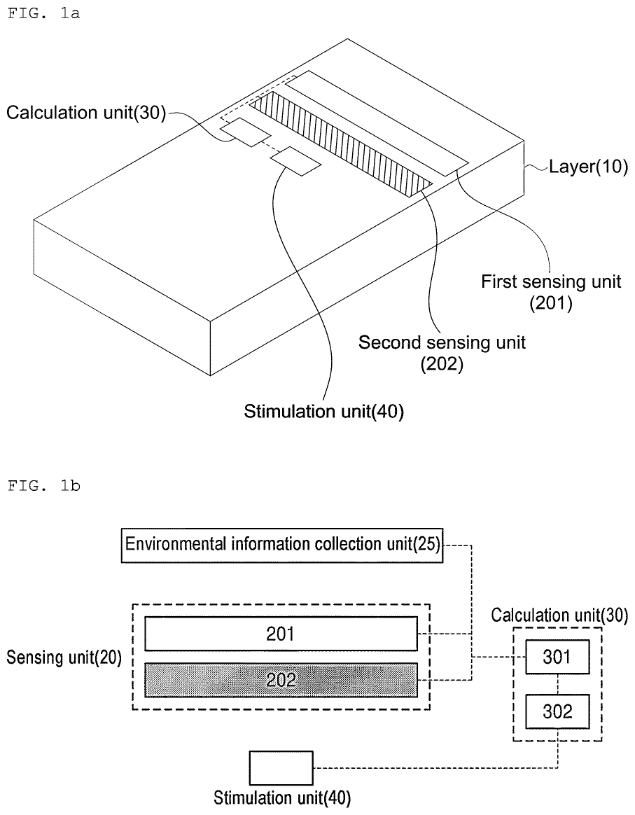

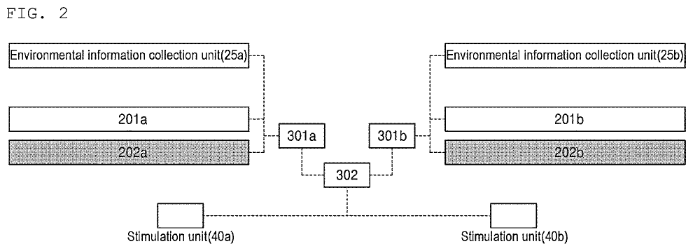

FIG. 1 schematically shows an unrestrained sleep improvement device according to the present disclosure. Referring to FIGS. 1A and 1B, an unrestrained sleep improvement device may largely include a layer 10, a sensing unit 20, an environmental information collection unit 25, a calculation unit 30, and a stimulation unit 40.

First, the layer 10 may be understood as a member having a receiving space in which the components to be described later can be arranged, and if a receiving space is provided, there is no limitation on the material or shape of the layer 10. The layer 10 may be, for example, a mattress, or may be any one of a plurality of surfaces constituting a mattress. Additionally, the layer 10 may be a mat that can be placed on a mattress, and may furthermore be a member made of wood or metal rather than cotton with a fiber material. In this manner, if the layer 10 has a predetermined receiving space, there is no limitation on the material or shape. However, in order to help understand the disclosure, in this detailed description, the explanation will be continued assuming that the layer 10 is a mattress.

Next, the sensing unit 20, which is provided on the layer, is configured to sense and acquire biometric information from a user. Specifically, the sensing unit 20 may be divided into two types, wherein the first sensing unit 201 may sense a vibration signal from the user, and the second sensing unit 202 may sense a pressure signal according to the user's movement.

The first sensing unit 201 may preferably be made of polyvinylidene fluoride (PVDF). The PVDF is a piezoelectric material that generates electricity when mechanical/physical stimulation is applied, and this property may be used to sense a vibration signal from a user lying on a mattress. The first sensing unit 201 ultimately senses a vibration to obtain the user's biometric information, particularly at least one of a ballistocardiogram, a respiratory state, or a movement state.

The second sensing unit 202 may preferably be implemented as an a force sensing resistor (FSR) array. The FSR is a component that converts a pressure value into a resistance value, and if a plurality of FSRs are provided in an array form, the user's posture may be estimated by referring to magnitudes of resistance values sensed when the user is lying on a mattress using the above property. In this detailed description, a set of values obtained by the second sensing unit 202 is defined as a pressure signal.

For reference, the first sensing unit 201 or the second sensing unit 202 in the above may have an elastic film added to the surface, and such elastic films may help to more accurately and sensitively sense signals that can be acquired from the user.

In addition, for reference, it should be understood that the sensing unit 20 is divided into the first sensing unit 201 and the second sensing unit 202 only for the purpose of helping to understand the disclosure, and is not intended to necessarily limit the embodiment of the present disclosure as described above. In particular, the sensing unit 20 in the present disclosure may be designed in various forms, and may be designed in a state where the first sensing unit 201 and the second sensing unit 202 are arranged side by side as shown in the drawing, or may be designed so as to allow vibration sensing and pressure sensing to be arranged on a single band-shaped member. It should be emphasized once again that there is no limitation on the design method of the sensing unit 20.

The environmental information collection unit 25 (see FIG. 1B) is configured to collect information items on the user's surrounding environment, and information items on the surrounding environment may include indoor temperature, noise, humidity, or illuminance. Appropriate means may be used to acquire respective environmental information items, for example, a thermometer, a microphone, a hygrometer, or a light meter.

The calculation unit 30 is configured to perform calculations to determine the user's sleep state from a vibration signal and/or a pressure signal acquired by the first sensing unit 201 and the second sensing unit 202. For reference, the calculation unit 30 may also be understood as a central processing unit. The central processing unit may also be referred to as a controller, a microcontroller, a microprocessor, a microcomputer, or the like. Furthermore, the central processing unit may be implemented by hardware or firmware, software, or a combination thereof, and configured to include an application specific integrated circuit (ASIC) or a digital signal processor (DSP), a digital signal processing device (DSPD), a programmable logic device (PLD), or a field programmable gate array (FPGA) when implemented using hardware, and configured with firmware or software to include a module, a procedure, a function or the like that performs the foregoing functions or operations when implemented using firmware or software. In addition, the calculation unit 30 may include a memory, and the memory may be implemented as Read Only Memory (ROM), Random Access Memory (RAM), Erasable Programmable Read Only Memory (EPROM), Electrically Erasable Programmable Read-Only Memory (EEPROM), flash memory, Static RAM (SRAM), a hard disk drive (HDD), a solid state drive (SSD) or the like.

The calculation unit 30 may extract meaningful signals through filtering from signals obtained by the sensing units, and may determine the user's current sleep state from at least one signal among the extracted signals or a combination of a plurality of meaningful signals. When dividing a person's sleep state, it may be largely divided into an awake state, a REM sleep state, and a non-REM sleep state, and it is known that the non-REM sleep state may be further divided into several stages (stage 1 pre-sleep, stage 2 light-sleep, stages 3 and 4 slow wave-sleep), wherein the calculation unit 30 may determine which stage among the awake state, REM sleep state, non-REM sleep state, or non-REM sleep state it is in based on signals sensed from the user.

Examining more specifically, the calculation unit 30 may extract at least one valid signal among a ballistocardiogram, a respiration signal, or a movement signal by applying a preset filter condition to the vibration signal (original signal) acquired from the first sensing unit 201, and may determine a current sleep state of the user based on those valid signals. In this case, the determination may vary depending on which valid signal is included within a specific range that has been set in advance, and the specific range that has been set in advance may be determined differently for each individual user. Describing more specifically, the calculation unit 30 may extract biometric signals such as a ballistocardiogram, a respiration signal, and a movement signal from a vibration signal (original signal), and the above extracted biometric signals may be used to obtain biometric information such as a heart rate, a respiration rate, a heart rate variability, a respiration variability, or a movement level, and those biometric information items may be used again to determine the user's sleep state. In this case, the determination of the sleep state may vary depending on which biometric information is included within a specific range that has been set in advance, and the specific range that has been set in advance may be determined differently for each individual user.

The calculation unit 30 may also estimate a sleeping posture of the user who is currently lying down based on the pressure signal acquired from the second sensing unit 202. That is, it may be possible to estimate from the pressure signal whether the user is lying on the right side, lying on the left side, lying upright, lying prone, or sitting on the layer 10. For example, a plurality of sensors may be arranged in the second sensing unit 202, and as shown in the drawing, the second sensing unit 202 may be provided across a width direction of the mattress (layer), and depending on the posture in which the user lies down, sensors that sense pressure and sensors that do not sense pressure will be distinguished, and also, among the sensors that sense pressure, the magnitudes of the pressure will be different from one another, and therefore, the user's posture may be estimated by looking at the pattern in which the sensors sense pressure. The user's sleeping posture estimated in this way may be used to determine the user's overall sleep state by referring to the vibration signal acquired by the first sensing unit 201 or the valid signal extracted therefrom.

Meanwhile, as may be seen in FIG. 1B, the calculation unit may be divided again into a first calculation unit 301 and a second calculation unit 302, wherein the first calculation unit 301 may receive signals from the sensing unit 20 and perform a function of primary processing on those signals, and the second calculation unit 302 may generate biometric information from signals previously processed by the first calculation unit 301 and perform a function of controlling the stimulation unit 40 based thereon. A signal processing function of the first calculation unit 301 may include a filtering function that extracts only signals in a specific band, and may also include a noise removal function that removes noise within the signal. If the first calculation unit 301 is focused on signal processing, the second calculation unit 302 may be more focused on signal interpretation and device control. That is, the second calculation unit 302 may produce an arbitrary determination result by performing signal interpretation through an algorithm from the acquired signal, and based thereon, may generate a control command to control other drive units in the sleep improvement device.

Lastly, the stimulation unit 40 is configured to generate stimulation to induce a change in the user's biometric signal according to the previously determined sleep state. The stimulation unit 40 may be implemented in various ways depending on the type of stimulation that can affect the user, and for example, the stimulation unit 40 may be implemented as an actuator that can generate vibration, a speaker that can generate sound, a heater that can generate heat, a cooler that can generate cold air, a fan that can generate wind, a light that can generate light, or any other component that can stimulate at least one of a person's senses.

The stimulation unit 40 is shown as being positioned and installed on the layer 10 in the drawing, but the stimulation unit 40 does not necessarily have to be on the layer 10, and may also be installed at an outside of the layer 10, that is, at a predetermined distance away from the layer 10. For example, assuming that the stimulation unit 40 is implemented as a speaker, the speaker may be installed on a wall of the room rather than on the mattress (layer) to output sound when the user is in a sleep state.

Meanwhile, the stimulation unit 40 may generate stimulation for the purpose of inducing a change in the user's biometric signal. In this case, the user's biometric signal may include various signals that can be sensed from the user, and the signals acquired by the first sensing unit 201 or the second sensing unit 202 may also be understood as a type of biometric signal. The meaning of inducing a change in biometric signal may ultimately refer to inducing the user's sympathetic or parasympathetic nerves to be activated through simulation in a way that improves the user's sleep quality. More specifically, the user's parasympathetic nerves may be activated to a desired state to allow the user to get a good quality of sleep, and the user's sympathetic nerves may be activated to allow the user to be induced to wake up at an appropriate time or at a set time (e.g., a set alarm time). In this case, the operation of the stimulation unit 40 may be performed differently depending on the user's sleep state determined by the calculation unit 30, and the calculation unit 30 may generate a series of control signals indicating which stimulation to apply towards which goal depending on the determined sleep state of the user, and transmits the signals to the stimulation unit 40, thereby allowing appropriate stimulation to be applied to the user. In this case, the goal may be set differently for each user, and in particular, if a degree of activation of the sympathetic or parasympathetic nerves has been measured in advance, a degree of activation during the time when the user is inferred to be in a deep sleep among the measured degrees of activation may be the goal. Alternatively, the goal may be a degree of activation that is already known from many studies, even if it has not been measured in advance.

For reference, in this detailed description, in order to help understand the disclosure, unless otherwise specifically stated, the stimulation unit 40 that generates vibration according to the actuator provided in the layer 10 is understood as a representative example.

In the above, an unrestrained sleep improvement device according to the present disclosure has been examined with reference to FIG. 1.

FIG. 2 shows another sleep improvement device according to the present disclosure, in which internal components of a sleep improvement device capable of acquiring biometric signals for two users are conceptually shown. Simply put, if FIGS. 1A and 1B are a sleep improvement device corresponding to a single mattress (layer) for one person, then FIG. 2 may be understood as a sleep improvement device corresponding to a double mattress (layer) for two people. The sleep improvement device according to FIG. 2 may be provided with sensing units 201a, 202a, an environmental information collection unit 25a, a calculation unit 301a, and a stimulation unit 40a on the left side of the layer 10, and may also be provided with sensing units 201b, 202b, an environmental information collection unit 25b, a calculation unit 301b, and a stimulation unit 40b on the right side of the layer 10 in the same manner. As a special feature, the second operation unit 302, that is, a component that interprets signals and generates control commands, may be provided with only one unit, and may be designed to receive signals from both the first calculation units 301a, 301b. In addition, in some cases, it is not necessary to have two environmental information collection units 25a, 25b, and only one may be provided around the layer 10 to share and utilize the collected environmental information.

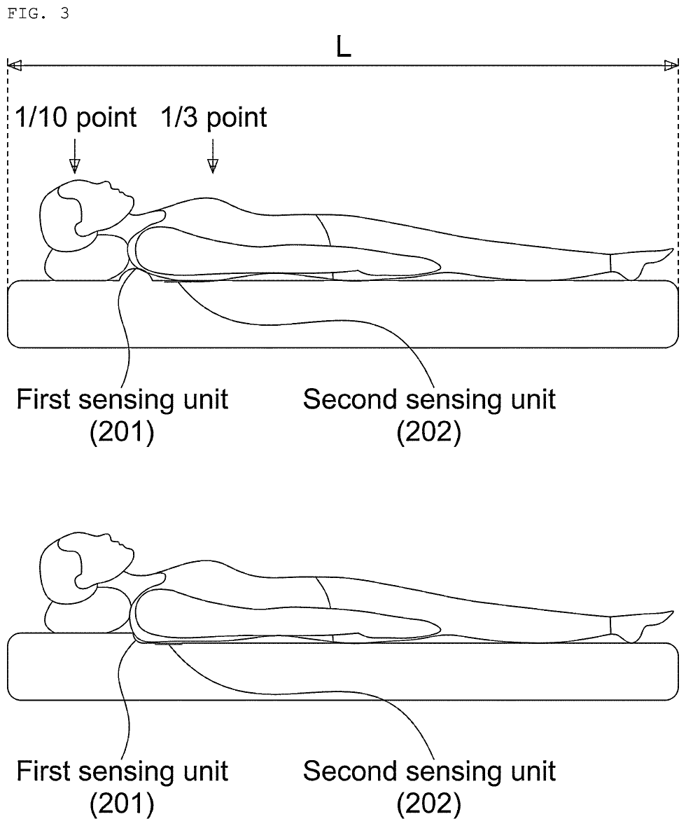

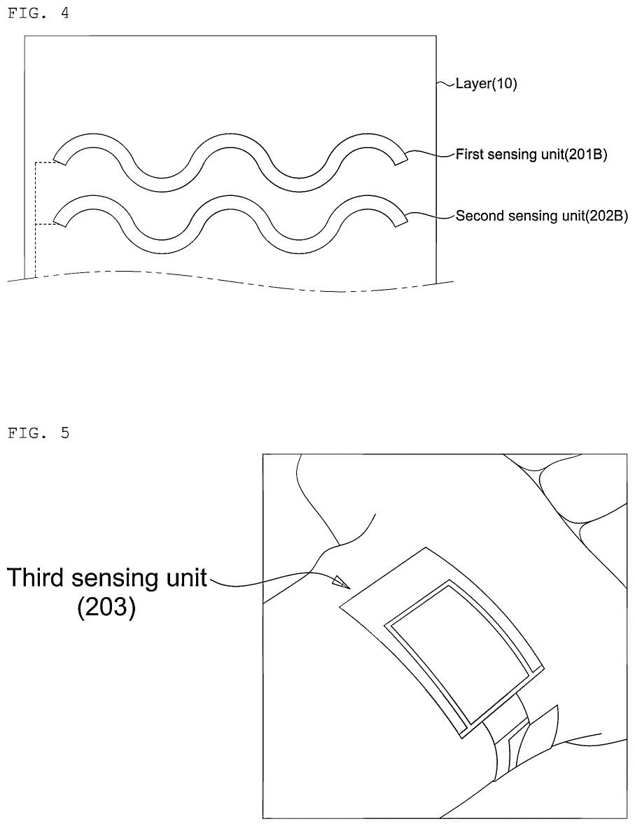

FIG. 3 shows a shape and arrangement of the sensing unit 20, but as can be seen in the drawing, the sensing unit 20 may not necessarily be implemented in a straight plane or band shape. In FIG. 1, it is shown that the first sensing unit 201 or the second sensing unit 202 is a band shape arranged side by side in a width direction on the layer 10, but in particular, in FIG. 3, it is shown that the shape of the first sensing unit 201 may not be a flat band shape.

Specifically, an upper drawing of FIG. 3 shows the first sensing unit 201 having a convex shape upward, and shows a configuration in which the second sensing unit 202 having a flat band shape is arranged therebelow. The reason why the shape of the first sensing unit 201 is convex is to more effectively sense a vibration signal from a lying user, and in particular, in order to obtain the user's ballistocardiogram, respiration signal, or the like, it is necessary to place the first sensing unit 201 as close as possible to an upper area of the user's shoulder or back, more preferably so as to be in direct contact therewith. For this purpose, in an unrestrained sleep improvement device according to the present disclosure, the first sensing unit 201 may be made to be convex in an upward direction based on the layer 10 on a plane. Additionally, the second sensing unit 202 may also be implemented in the same shape for the purpose of effectively obtaining a pressure signal from the user. Meanwhile, when observing the upper drawing of FIG. 3 in more detail, a convex shape of the first sensing unit 201 may induce the user to set a sleeping location so as to allow the convex shape to be located at his or her neck area. In general, when looking at a person lying down from the side, a line connecting the head, neck, and shoulders includes a curve as shown in the drawing, and the user may determine a sleeping location so as to allow the convex shape of the first sensing unit 201 to be located along this curve. For example, from the user's point of view, he or she may visually check a slightly convex portion on the mattress (layer) and then adjust the pillow and sleeping location so as to allow his or her neck to located on that portion. Alternatively, when the first sensing unit 201 is provided inside a cover or the mattress (layer) and therefore it is difficult for the user to visually estimate its location, a guide line that allows the user to estimate the location of the first sensing unit 201 may be marked on a surface of the mattress (layer) so as to allow the user to determine a sleeping location by looking at it. In order to allow the location of the first sensing unit 201 to be identified, a mattress cover with a guide line may be manufactured and provided separately, and the user may determine his or her sleeping location through this.

Meanwhile, a lower part of FIG. 3 shows another shape of the first sensing unit 201. Here, it can be confirmed that the first sensing unit 201 is implemented in a soft curved ‘L’ shape, and it can be seen that the first sensing unit 201 of this shape is positioned to adhere closely to the shoulder line of the user lying down when viewed from the side. The first sensing unit 201 is implemented in such a curved ‘L’ shape is to effectively sense a vibration signal from the user, and as in an upper drawing of FIG. 3, the user may be guided to determine a sleeping location according to the shape of the first sensing unit 201. In this case, an outer shape of the mattress (layer), especially a line viewed from the side, may be formed into a concave shape from a specific portion according to a shape of the first sensing unit 201, and the user may be naturally guided to adjust a location of the pillow and his or her sleeping location along this concave shape. In addition, as in the description of the upper drawing of FIG. 3, in cases where it is difficult for the user to visually estimate the location of the first sensing unit 201 because it is provided inside a cover of the mattress (layer), a guide line may be marked on a surface of the mattress (layer) to estimate the location of the first sensing unit 201, or a cover of the mattress (layer) with a guide line indicated separately, thereby enhancing user convenience.

Meanwhile, the locations of the first sensing unit 201 and the second sensing unit 202 may preferably be provided within an area of 1/10 to ⅓ from the top based on a vertical length of the layer. The above range is determined by considering an upper area of the user's shoulders and back when the user is lying down on the layer 10, that is, an area close to or in direct contact with the sensing units.

In the above, the shape and location of the sensing unit have been examined with reference to FIG. 3.

FIG. 4 is a drawing showing a plan view of the layer 10 to explain that the first sensing unit 201B and the second sensing unit 202B can also be implemented in a wave shape.

As mentioned above in FIG. 3, the sensing units may be implemented in a convex or concave shape, and herein, the sensing units in a wave shape with a curve on a plane as shown in the drawing will be described. The wave-shaped sensing unit may have technical effects in two major aspects, the first of which is that it can increase the efficiency of sensing. In general, the width of the mattress (layer) is standardized and fixed, and when the sensing unit is implemented in a general band shape, only a limited number of detailed elements (sensors) constituting the sensing unit may be arranged, but, when it is manufactured in a wave shape as in FIG. 4, even with the same width of the mattress (layer), a greater number of sensors or a wider user contact surface area may be created, thereby increasing the efficiency of sensing. In particular, when first manufacturing a sleep improvement device, the sensing effect of the sensors may be improved by adjusting a wave valley and crest width during the design process. A second technical effect is that it may increase the durability of the sensor. Compared with a band-shaped sensing unit, a wave-shaped sensing unit with many curves may have better properties in terms of flexibility of the member, and in particular, considering the characteristics of a product such as a mattress or bed, the pressure applied by the user's movement may put a considerable burden on the sensing unit, so the durability of the sensing unit may be further increased by selecting a wave shape that can add flexibility. The band-shaped sensing unit may also increase flexibility depending on the material selected, but in this embodiment, it is assumed that the band-shaped sensing unit and the wave-shaped sensing unit are made of the same material, and it is intended to emphasize that the wave-shaped sensing unit shows better performance in terms of flexibility.

On the other hand, FIG. 4 shows a configuration in which both the first sensing unit 201B and the second sensing unit 202B are implemented in a wave shape, and as in the case of a band shape, the two sensing units may be arranged with a predetermined gap above and below. It is not necessary for the first sensing unit 201B and the second sensing unit 202B to have waves of the same shape, and, for example, the two sensing units may include curves with different curvatures or may have different lengths. Additionally, either of the two sensing units may retain a conventional band shape. For example, the first sensing unit 201B may be implemented in a band shape while only the second sensing unit 202B may be implemented in a wave shape. Alternatively, furthermore, the first sensing unit 201B may be implemented in a convex shape or a concave shape as in FIG. 3 above, and the second sensing unit 202B may be implemented in a wave shape. In this way, the sensing units that can be provided on the layer 10 may be manufactured with different shapes according to the designer's intention.

FIG. 5 shows an embodiment of a third sensing unit 203 in an unrestrained sleep improvement device according to the present disclosure. The third sensing unit 203 may be of a type that can be adhered or attached to a user's skin as shown, and may preferably be a surface-shaped sensing unit. The third sensing unit 203 may include an adhesive layer that can be adhered to a surface of the user's skin, and may also include a sensor strip for sensing a signal on the surface, and other elements that can operate as a sensing unit. The third sensing unit 203 may be provided with a communication means (e.g., Bluetooth) capable of short-distance communication to transmit the sensed signal to the previously mentioned calculation unit 30. In this case, it is assumed that another communication means capable of receiving a signal from the third sensing unit 203 is provided on the layer 10.

The third sensing unit 203 may be preferably attached around the location of the user's heart to sense a heart rate or vibration signal according to heart beat. The first sensing unit 201 may be also used to identify the user's sleep state by acquiring a vibration signal, but the first sensing unit 201 may not be able to make direct contact with the user's skin due to its structure, and thus the acquired data may be inaccurate, and data acquired from the third sensing unit 203 may be used to compensate therefor. For example, even if the first sensing unit 201 fails to sense the user's vibration signal due to some circumstances, the sleep state may be analyzed and determined using the signal received from the third sensing unit 203. Alternatively, when an error between a valid signal extracted from a signal received from the first sensing unit 201a and a signal received from the third sensing unit 203 corresponding to the valid signal is outside a preset range (e.g., more than 20%), the signal acquired by the third sensing unit 203 may be utilized for sleep state analysis and identification while excluding the valid signal extracted from the first sensing unit 201.

Meanwhile, in the drawing, the third sensing unit 203 is shown only as an adhesive type, but in some cases, the third sensing unit 203 may also be implemented as a band type. That is, when the third sensing unit 203 has a form in which the sensing strip or sensing element can be in direct contact with the user's skin surface, it may be defined as the third sensing unit 203 regardless of whether it is an adhesive type or a band type.

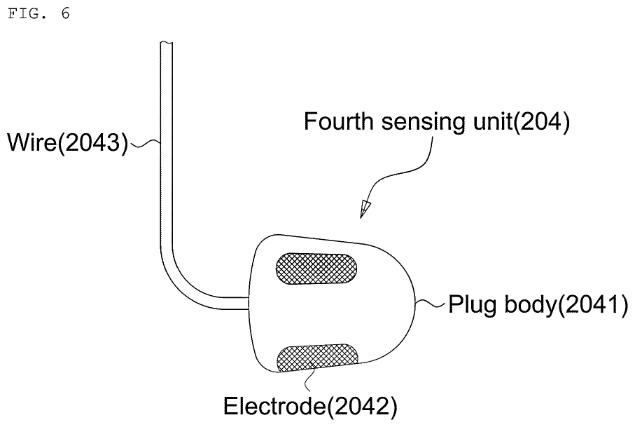

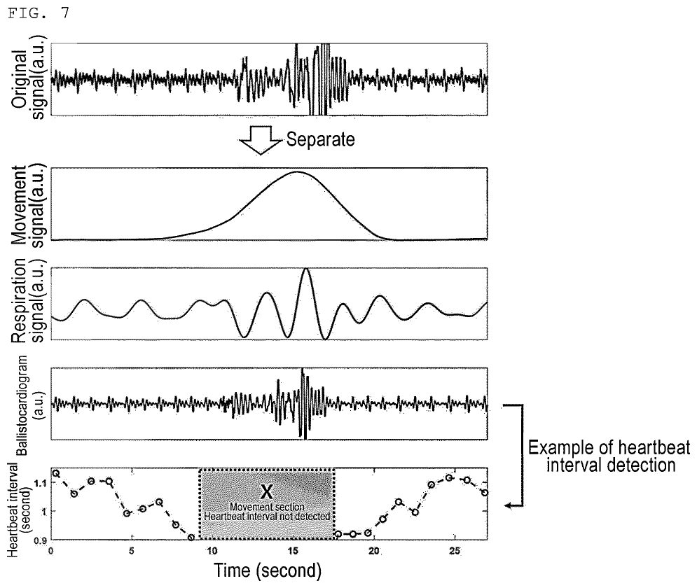

FIG. 6 shows a fourth sensing unit 204 that can be included in a sleep improvement device. The fourth sensing unit 204 may be an earplug-type sensing unit, and may be used by a user while wearing it in his or her ear while sleeping. The fourth sensing unit 204 is configured to detect ear-EEG, which is a signal (electroencephalogram) that allows brain activity to be identified through a minute voltage change that can be measured from the skin inside the ear. The fourth sensing unit 204 may include a plug body 2041 that is inserted into the ear, and an electrode 2042 provided in the plug body to sense a minute voltage change, and may additionally include a wire 2043 for transmitting a signal to the outside or receiving power from the outside. The fourth sensing unit 204 may also be implemented in a wireless manner, in which case it may be provided with a wireless communication means to provide the calculation unit 30 with data necessary for analyzing and determining a sleep state. That is, the calculation unit 30 may identify the user's sleep state by utilizing at least one of the vibration signal acquired from the first sensing unit 201, the pressure signal acquired from the second sensing unit 202, the signal acquired from the third sensing unit 203, and the ear-EEG signals acquired from the fourth sensing unit 204.

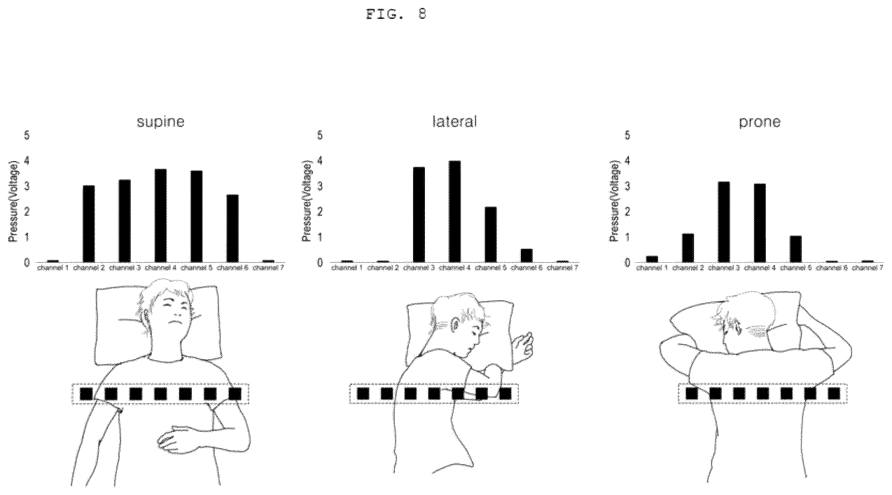

FIG. 7 shows a process in which the calculation unit 30 extracts various valid signals from a vibration signal acquired by the first sensing unit 201. Referring to FIG. 7, a signal sensed by the first sensing unit 201 may have the same waveform as an original signal, and the signal sensed in this manner may be directly transmitted to the calculation unit 30. The calculation unit 30 may extract at least one valid signal among a ballistocardiogram, a respiration signal, or a movement signal from the above original signal. What is meant by extracting a valid signal is that the calculation unit 30 can obtain a signal according to a filter condition by filtering the original signal. That is, the calculation unit 30 may obtain valid signals by setting the characteristics of the valid signal to be extracted as a filter condition and consequently applying the filter condition to the original signal.

For reference, a lower part of FIG. 7 shows a configuration in which the calculation unit 30 detects a heartbeat interval from a ballistocardiogram, and in this manner, the calculation unit 30 may extract a specific signal from the original signal acquired by the sensing unit 20 and then derive another meaningful signal therefrom, thereby determining the user's sleep state or autonomic nervous system activity state. Meanwhile, when detecting the heartbeat interval, the calculation unit 30 may be designed to detect the heartbeat interval only during other times, excluding the time during which the user's movement is estimated to occur. For example, during a time when a movement signal among signals separated from the original signal is below a preset value, or during a time when a magnitude value of the ballistocardiogram exceeds a preset value, the user's movement may be determined to be present so as not to detect the heartbeat interval.

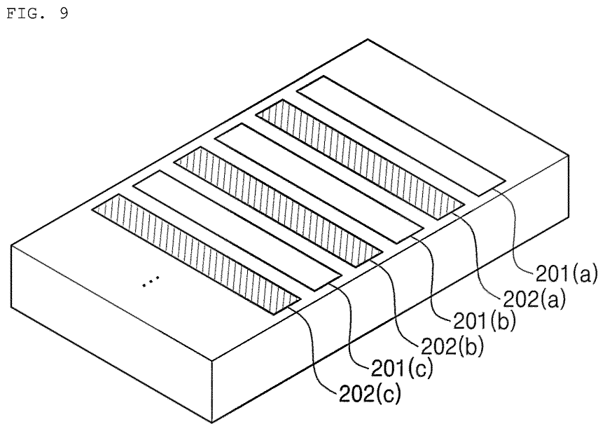

FIG. 8 shows a process in which the calculation unit 30 estimates a user's sleeping posture from a pressure signal acquired by the second sensing unit 202. Referring to FIG. 8, the signal sensed by the second sensing unit 202 may have values such as those shown at the top of the drawing, and the signal sensed in this manner may be analyzed by the calculation unit 30 and utilized to determine the user's current posture. Specifically, based on a magnitude value of the pressure signal over time, the calculation unit 30 may determine whether the user is lying supine, lying on the right or left side, lying with his or her arms down, or sitting. For reference, the calculation unit 30 may store pressure signals for each user's posture in advance, and analyze the user's sleeping posture in real time by referring to those pressure signals. Referring to FIG. 8, it can be seen that pressure signals are sensed only from channels 2 to 6 among the sensors from channels 1 to 7 in a supine position, pressure signals are sensed from channels 3 to 6 but signal values of channels 5 and 6 are gradually lowered in a left-lying position (lateral_left), and valid signal values are shown from channels 1 to 5, but signal values of channels 3 and 4 have a relatively larger gap compared to the remaining channels when the user is in a prone position.

Meanwhile, the calculation unit 30 may not only extract valid signals from the original signal, but also perform a process of determining the user's sleep state from those signals. As briefly mentioned above, the sleep state may be divided into an awake state, a REM sleep state, and a non-REM sleep state, and the non-REM sleep state may be further divided into several stages, wherein the calculation unit 30 can determine a current sleep state of the user by using at least one of several types of valid signals that can be extracted from the original signal. For example, when the ballistocardiogram shows peak-to-peak values from a1 to a2 and the respiration shows peak-to-peak values from b1 to b2, the user's sleep state may be determined to be in a REM sleep state, or when one cycle of the respiration signal is 5 seconds or longer, the user may be determined to be in slow-wave sleep state, or when the movement signal shows different values 4 or more times within 10 seconds, the user may be determined to be in an aroused state, and the like. Alternatively, a process of determining that a person is in an aroused state when average movement information extracted from a movement signal is above a specific magnitude, determining that the person is in a slow-wave sleep state when a heart rate interval is extracted from a ballistocardiogram and a degree of fluctuation of the heart rate interval is quantified in a time or frequency domain and a value thereof is above/below a specific magnitude, determining that the person is in a REM sleep state when a breathing interval is extracted from a breathing signal and a degree of fluctuation in the breathing interval is quantified in a time or frequency domain and a value thereof is above/below a specific magnitude, or the like, may be carried out.

In addition, the calculation unit 30 may record a correlation between a vibration signal acquired by the first sensing unit 201 and a pressure signal acquired by the second sensing unit 202 per unit time to create a database. When an arbitrary user is present, several signals may be acquired from the user, and there may be a characteristic correlation between them, wherein the calculation unit 30 of the present disclosure may store such a correlation in a database per unit time so as to more accurately determine a sleep state of an individual user. A correlation between signals may be defined by various formulas, and there is no particular limitation thereon. In addition, signals acquired by the first sensing unit 201 and the second sensing unit 202, as well as signals acquired by the third sensing unit 203 and the fourth sensing unit 204, may be utilized to create a database on such a correlation. For example, a first correlation between a ballistocardiogram and a pressure signal extracted from a vibration signal may be calculated per unit time and stored in a database, or a second correlation between a respiration signal extracted from a vibration signal and an ear-EEG signal may also be calculated per unit time and stored in a database. The correlations that have been databased in this manner may be used as reference data when the calculation unit 30 determines a sleep state of a specific user, and may make it possible to inquire what sleep state was determined in the past under the condition, especially when a specific correlation was shown at a specific time period, thereby increasing the calculation accuracy and speed of the calculation unit 30.

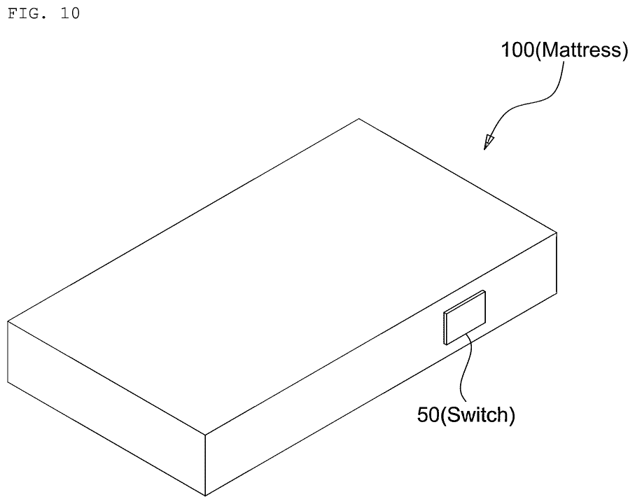

FIG. 9 shows a configuration in which a plurality of sensing units are provided on the layer 10. In the above, FIG. 1 or 3 shows a configuration in which the first sensing unit and the second sensing unit are arranged one by one, but if necessary, a plurality of sensing units may be arranged as in FIG. 9. When the plurality of sensing units are arranged, more vibration signals and pressure signals may be acquired from a user, and in particular, pressure signals may be obtained throughout an entire area of the layer 10 so as to more accurately identify a sleeping posture.

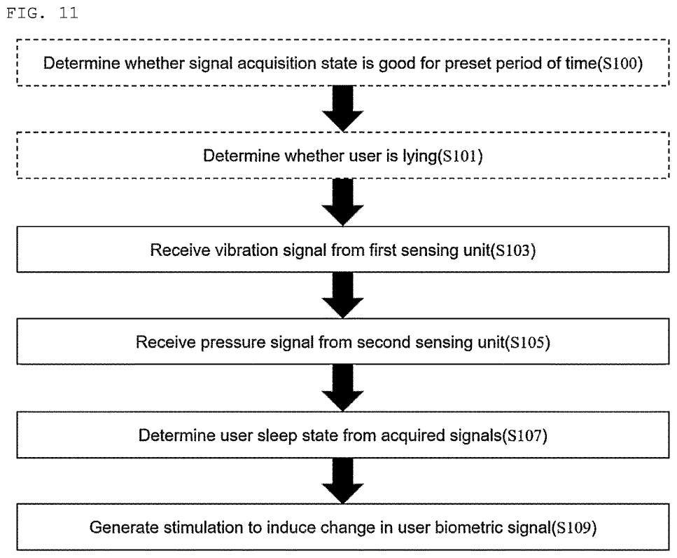

FIG. 10, which is a configuration of a sleep improvement device according to another embodiment of the present disclosure, may have an input pad 50 provided on a side surface of a mattress 100. In the above, it has been described that the sleep improvement device is provided with a stimulation unit 40, and the stimulation unit may generate stimulation that can induce a biometric signal depending on the user's sleep state, but the operation of the stimulation unit 40 must be stopped in some cases, and as a result, in the present disclosure, the operation of the stimulation unit 40 may be easily and intuitively stopped by placing an input pad 50 on a side surface of the mattress 100. For example, when the calculation unit 30 incorrectly determines the user's sleep state and generates long-cycle vibratory stimulation even though the user has not yet fallen asleep, the user may stop the vibration by pressing the input pad 50 on the side surface of the mattress, and at the same time, initialize the operation of the sensing units to identify the user's sleep state again from the beginning. Additionally, the stimulation unit 40 may generate an alarm stimulus to wake up the user in the morning, and in this case, the user may stop the alarm stimulus by pressing the input pad 50.

Meanwhile, the input pad 50 may be provided at a location other than the side surface of the mattress, and may be located anywhere if it is a location that can be easily reached by the user's hand or a location that can be easily reached by the user's feet. In addition, the input pad 50 may be designed to be large in size so as to allow a user who is not fully awake to easily find and press it, and for example, may be designed to be large in size, such as a square with a one-side length of 10 cm or more, or a square with a one-side length of 50% or more in a thickness of the mattress.

FIG. 11 sequentially shows a method of improving a user's sleep according to another embodiment of the present disclosure. Referring to FIG. 11, a sleep improvement method may first include determining whether a user is lying on the layer 10 (S101), receiving a vibration signal from a first sensing unit (S103), and then receiving a pressure signal from a second sensing unit (S105), and may then include determining a sleep state or an autonomic nervous system state of the user from the signals acquired by the first sensing unit and the second sensing unit in parallel (S107), and generating stimulation to induce a change in the user's biometric signal according to the sleep state or autonomic nervous system state (S109). That is, referring to FIG. 11, the sleep improvement method according to the present disclosure may determine the user's sleep state or autonomic nervous system state (parasympathetic nerve, sympathetic nerve activity state) when the user's biometric signal is sensed from the sensing unit 20, and may also generate stimulation for inducing a change in the user's biometric signal to a specific state by referring to the sensed biometric signal.

On the other hand, the steps S107 and S109 may be sequentially carried out from step S107 to step S109 rather than in parallel subsequent to step S105. In other words, in the sleep improvement method according to the present disclosure, the user's sleep state may be identified from a signal acquired from the user, and then stimulation may be generated based on the identified sleep state.

Meanwhile, in the method of improving a sleep, first of all, an additional step of checking whether a vibration signal or a pressure signal is well sensed from the user may be additionally included. The first sensing unit and the second sensing unit mentioned in the present disclosure are components, which are both fixedly installed on the layer 10, and the user cannot arbitrarily adjust their locations, and for that reason, the user needs to identify where he or she should lie on the layer 10 so as to accurately analyze his or her sleep state. Step S100 is determining whether a signal acquisition state of the first sensing unit or the second sensing unit is good for a preset period of time, and specifically, may be understood as finding a location where signal acquisition is the best by having the user repeatedly lie down on the layer 10 several times for a predetermined period of time. For example, the user may find an appropriate location by repeatedly changing locations according to a given guide, such as lying in location 1 for 20 seconds, then lying in location 2 for another 20 seconds, and the like. Among the above steps, steps S100 and S101 may not necessarily be required.

In the above, an unrestrained sleep improvement device according to the present disclosure and a sleep improvement method using the same have been examined. Meanwhile, the present disclosure is not limited to the foregoing specific embodiments and application examples, it will be of course understood by those skilled in the art that various modifications may be made without departing from the gist of the present disclosure as defined in the following claims, and it is to be noted that those modifications should not be understood individually from the technical concept and prospect of the present disclosure.

In particular, configurations that implement the technical features of the present disclosure included in the block diagrams and flowcharts shown in the drawings attached to this specification represent logical boundaries between the configurations. However, according to an embodiment of software or hardware, the shown configurations and functions thereof are executed in the form of stand-alone software modules, monolithic software structures, codes, services, and combinations thereof, and the functions may be implemented by being stored in a medium executable on a computer provided with a processor capable of executing the stored program codes, instructions, and the like, and therefore, all of these embodiments should also be regarded as falling within the scope of the present disclosure.

Accordingly, the accompanying drawings and technologies thereof describe the technical characteristics of the present disclosure, but should not be simply inferred unless a specific array of software for implementing such technical characteristics is clearly described otherwise. That is, the aforementioned various embodiments may be present, and may be partially modified while having the same technical features as those of the present disclosure, and thus such modified embodiments should also be regarded as falling within the scope of the present disclosure.

Furthermore, the flowchart describes operations in the drawing in a specific sequence, but has been shown to obtain the most preferred result, and it should not be understood that such operations must be carried out in the specific sequence or sequential sequence shown, or that all shown operations must be carried out. In a specific case, multi-tasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

1. An unrestrained sleep improvement device, the device comprising:
at least one layer comprising a plurality of components;
a first sensing unit provided on the layer to acquire a vibration signal from a user;
a second sensing unit provided on the layer to acquire a pressure signal according to the user's movement;
a calculation unit that determines the user's sleep state or autonomic nervous system state from the signals acquired by the first and second sensing units; and
a stimulation unit that generates stimulation to induce a change in the user's biometric signal according to the sleep state or autonomic nervous system state.

2. The device of claim 1, wherein at least one of the first sensing unit or the second sensing unit is provided within an area of 1/10 to ⅓ from the top based on a vertical length of the layer.

3. The device of claim 1, wherein at least one of the first sensing unit or the second sensing unit comprises a curved surface that is curved in an upward or downward direction of the layer.

4. The device of claim 1, wherein the first or second sensing unit comprises a wave-shaped belting member and a plurality of sensors arranged on the member.

5. The device of claim 1, wherein the calculation unit processes a vibration signal acquired by the first sensing unit to extract a plurality of valid signals, and generates biometric information on the user from the extracted valid signals.

6. The device of claim 1, wherein the calculation unit records a correlation between the vibration signal acquired by the first sensing unit and the pressure signal acquired by the second sensing unit per unit time to create a database.

7. The device of claim 1, wherein the unrestrained sleep improvement device is a mattress, the device further comprising:
a touch-type input pad, which is provided on a side surface of the mattress to receive an touch input from the user.

8. A method of improving a user's sleep using an unrestrained sleep improvement device, the method comprising:
receiving a vibration signal from a first sensing unit;
receiving a pressure signal from a second sensing unit;
determining the user's sleep state or autonomic nervous system state from the signals acquired by the first and second sensing units; and
generating stimulation to induce a change in the user's biometric signal according to the sleep state or autonomic nervous system state.
